(19) United States
(12) Patent Application Publication
(10) Pub. No.: US 2006/0211747 A1
(43) Pub. Date: Sep. 21, 2006

(54) Methods of screening for compounds which inhibit the activity of Cdc34 in a zinc-mediated manner and compounds obtained by this method

(76) Inventors: Pascal Furet, Thann (FR); Francesco Hofmann, Basel (CH); Edgar Jacoby, Basel (CH); Joseph Schoepfer, Riehen (CH)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 11/226,786

(22) Filed: Sep. 14, 2005

(30) Foreign Application Priority Data
Sep. 15, 2004 (GB) 0420519.1

(51) Int. Cl. A61K 31/43

(52) U.S. Cl. 514/366

(57) ABSTRACT

The present invention relates to methods of searching/screening for compounds which inhibit the activity of the ubiquitin conjugating enzyme Cdc34 in a zinc-mediated manner, to compounds obtainable by such a method, to a method of treating mammalian subjects with such compounds, to pharmaceutical compositions containing such compounds, to the use of such compounds for the treatment of diseases which respond to an inhibition of the activity of Cdc34 such as proliferative diseases, especially tumor diseases, and to the use of such compounds for the preparation of such pharmaceutical compositions for the treatment of such diseases.

The present invention relates to methods of searching/screening for compounds which inhibit the activity of the ubiquitin conjugating enzyme Cdc34 in a zinc-mediated manner, to compounds obtainable by such a method, to a method of treating mammalian subjects with such compounds, to pharmaceutical compositions containing such compounds, to the use of such compounds for the treatment of diseases which respond to an inhibition of the activity of Cdc34 such as proliferative diseases, especially tumour diseases, and to the use of such compounds for the preparation of such pharmaceutical compositions for the treatment of such diseases.

### DESCRIPTION OF THE FIGURES

FIG. 1: This Figure shows the sequence alignment of human UBC3 with other E2 enzymes with known X-ray structure. The SwissProt sequences // PDB structures are: UBC3_HUMAN P49427 homo sapiens (human Cdc34); UBC7_YEAST Q02159 saccharomyces cerevisiae // 2UCZ; UBC1_ARATH P25865 arabidopsis thaliana (mouse-ear cress) // 2AAK; UBC2_YEAST P06104 saccharomyces cerevisiae // 1AYZ; UBC4_YEAST P15731 saccharomyces cerevisiae // 1QCQ; UBCB_SPISO Q95044 spisula solidissima (atlantic surf-clam) // 2E2C; UBCI_HUMAN P50550 homo sapiens and mus musculus // 1A3S and U9B; and UBC7_HUMAN P51966 homo sapiens and mus musculus // 1C4Z. The secondary structure is indicated for the UBC3 homology model in the top line and is calculated within Sybyl ProTable: B—residue in isolated β-strand; E—extended β-sheet; H—α helix; G—3/10 helix; T—hydrogen bond turn. § indicates inserted residues relative to the UBC7 template structure; no structure is proposed for these residues what leaves an open gap within the structure at this place.

FIG. 2: Clustal alignment of the region containing the active site cysteine (*) of 25 canonical E2s. The histidine residue (#), putatively implicated in the coordination of zinc, is peculiar to Cdc34 and its most closely related E2s.

FIG. 3: The inhibition of Cdc34 thioester formation measured in the presence (right panel) of absence (left panel) of 50 μM phenanthroline is plotted for the indicated compounds. The IC₅₀ values estimated from this curves are indicated on top of each plot.

### DESCRIPTION OF THE INVENTION

Ubiquitin/ubiquitin-like mediated signaling regulates a variety of biological processes. Modification of substrate proteins with ubiquitin or ubiquitin-like molecules requires the concerted action of an ubiquitin activating enzyme (E1), various ubiquitin conjugating enzymes (E2) and several ubiquitin ligases (E3). During this process, thioester intermediates are formed between the carboxy-terminus of ubiquitin/ubiquitin-like molecules and a cysteine residue at the active site of E1, E2s, and some E3s (for review see Ciechanover A., EMBO J. 17, 7151-7160, 1999). Thus, one possibility to interfere with this process is the development of small molecule inhibitors that prevent a specific E2 or E3 to form the thioester intermediate.

The ubiquitin conjugating enzyme Cdc34 (SwissProt P49427) is considered an interesting target in the context of oncology, because it was shown to be involved in the regulation of cell cycle and the ubiquitin-dependent degradation of the cyclin-dependent kinase inhibitor p27, a tumor suppressor, whose degradation is often enhanced in tumors (Sgambato et al., J. Cell. Physiol. 183, 18-27, 2000; Slingerland J. and Pagano M., J. Cell. Physiol. 183, 10-17, 2000).

Analysis of the three-dimensional organization of the Cdc34 molecule, based on a homology model constructed with the help of the crystal structure of the related yeast ubiquitin conjugating enzyme Ubc7 (Cook et al., Biochemistry 36, 1621-1627, 1997) revealed the presence of a cysteine-histidine pair (Cys93 and His98 of human Cdc34; SwissProt P49427) at the active site of Cdc34. It has now been found that Cdc34 contains zinc being bound at the active site of Cdc34 via this cysteine-histidine pair.

Accordingly, the present invention provides a method of searching for compounds which inhibit the activity of the ubiquitin conjugating enzyme Cdc34 in a zinc-mediated manner, which method comprises identifying compounds capable of coordinating zinc together with the active site cysteine-histidine residues of Cdc34 using a computer assisted molecular modeling (CAMM)-based approach.

The present invention further also provides a method of screening for compounds which inhibit the activity of the ubiquitin conjugating enzyme Cdc34 in a zinc-mediated manner, which method comprises determining the capacity of a compound to inhibit the activity of Cdc34 in the presence of zinc [Cdc34(+)], determining the capacity of the compound to inhibit the activity of Cdc34 that is depleted of zinc [Cdc34(−)], comparing both capacities and selecting compounds which inhibit the activity of Cdc34(+) to a greater extent than Cdc34(−). This method can be used for example to screen compound libraries for zinc-mediated Cdc34 inhibitors or to confirm the ability of a compound, found by e.g. the CAMM-based approach of the method of the preceding paragraph, to inhibit Cdc34 in a zinc-mediated manner. The capacities of a compound to inhibit Cdc34(+) and Cdc34(−) can be determined for example by determining its IC₅₀ values for the inhibition of Cdc34(+) and Cdc34(−), e.g. by using the thioester assays as described below. Preferably, a compound which inhibits the activity of Cdc34 in a zinc-mediated manner exhibits a ratio between its IC₅₀ value for the inhibition of Cdc34(+) and its IC₅₀ value for the inhibition of Cdc34(−) of more than 2/1, especially of more than 10/1, most preferably of more than 50/1.

In a further embodiment, the present invention relates to a compound obtainable by the method of the preceding paragraph, or a pharmaceutically acceptable salt of such a compound (Compound of the Invention). Such a compound is in particular a compound selected from the group consisting of (3,5-dimethyl-pyrazol-1-yl)-(3-methyl-imidazo[2,1-b]thiazol-6-yl )-methanone, (3,5-dimethyl-pyrazol-1-yl)-imidazo[2,1-b]thiazol-6-yl-methanone and (3,5-dimethyl-pyrazol-1-yl)-(3-propyl-imidazo[2,1-b]thiazol-6-yl)-methanone, or a pharmaceutically acceptable salt of such a compound.

The present invention further also relates to a Compound of the Invention, or a pharmaceutically acceptable salt of such a compound, for use in a method for the therapeutic treatment of the human or animal body.

In another embodiment, the present invention relates to a method of treating a mammalian subject, especially a human, suffering from a disease which responds to an inhibition of the activity of the ubiquitin conjugating enzyme Cdc34, which method comprises administering to the subject suffering from said disease a Compound of the Invention, or a pharmaceutically acceptable salt of such a compound, in an amount effective against said disease. A disease which responds to an inhibition of the activity of the ubiquitin conjugating enzyme Cdc34 is, in the context of this disclosure, especially a proliferative, preferably a tumour, disease which responds to an inhibition of the activity of the ubiquitin conjugating enzyme Cdc34. Such diseases include for example colon, lung, breast and prostate cancers.

The present invention further also relates to the use of a Compound of the Invention, or a pharmaceutically acceptable salt of such a compound, in the treatment of a disease which responds to an inhibition of the activity of the ubiquitin conjugating enzyme Cdc34.

The Compounds of the Invention, or a pharmaceutically acceptable salt thereof, can be administered as such or in the form of pharmaceutical compositions, prophylactically or therapeutically, preferably in an amount effective against the diseases mentioned herein, to a warm-blooded animal, especially a human, requiring such treatment, the compounds especially being used in the form of pharmaceutical compositions. In the case of an individual having a bodyweight of about 70 kg the daily dose administered is from approximately 0.1 g to approximately 5 g, preferably from approximately 0.5 g to approximately 2 g, of a Compound of the Invention.

Accordingly, the present invention also relates to a pharmaceutical composition comprising a Compound of the Invention, or a pharmaceutically acceptable salt of such a compound, together with a pharmaceutically acceptable carrier. The invention further also relates to a pharmaceutical composition for the treatment of a disease which responds to an inhibition of the activity of the ubiquitin conjugating enzyme Cdc34 in a mammalian subject, especially a human, comprising, in a dose effective against said disease, a Compound of the Invention, or a pharmaceutically acceptable salt of such a compound, together with a pharmaceutically acceptable carrier.

The present invention also relates to the use of a Compound of the Invention, or a pharmaceutically acceptable salt of such a compound, for the preparation of a pharmaceutical composition for use in the treatment of a disease which responds to an inhibition of the activity of the ubiquitin conjugating enzyme Cdc34.

Pharmaceutical compositions for enteral administration, such as nasal, buccal, rectal or, especially, oral administration, and for parenteral administration, such as intravenous, intramuscular or subcutaneous administration, to warm-blooded animals, especially humans, are especially preferred. The pharmaceutical compositions contain the active ingredient alone or, preferably, together with a pharmaceutically acceptable carrier. The dosage of the active ingredient depends upon the disease to be treated and upon the species, its age, weight, and individual condition, the individual pharmacokinetic data, the mode of administration, and other factors known to the person skilled in the art.

The pharmaceutical compositions comprise from approximately 1% to approximately 95% active ingredient, single-dose administration forms comprising in the preferred embodiment from approximately 20% to approximately 90% active ingredient and forms that are not of single-dose type comprising in the preferred embodiment from approximately 5% to approximately 20% active ingredient. Unit dose forms are, for example, coated and uncoated tablets, ampoules, vials, suppositories or capsules.

The pharmaceutical compositions of the present invention are prepared in a manner known per se, for example by means of conventional mixing, granulating, coating, dissolving or lyophilising processes.

If desired, said pharmaceutical compositions may also contain further active components, such as other chemotherapy drugs, and/or may be used in combination with known therapeutic processes, for example the administration of hormonal medicines or other chemotherapeutic drugs, or radiation.

### 1. Computer Assisted Molecular Modeling (CAMM)-based Approach

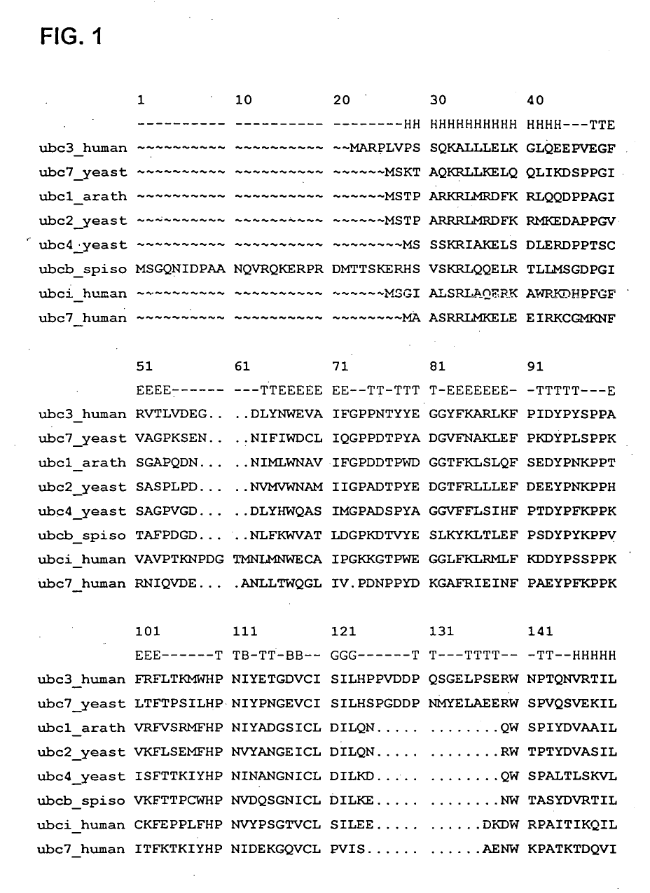

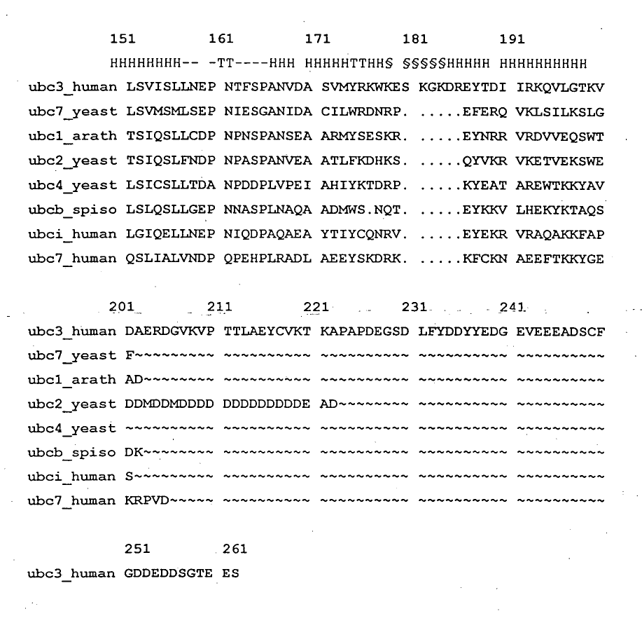

i) Homology modeling of human Cdc34 is done with WhatIf99. The Protein Data Bank (PDB) crystal structure 2UCZ from Ubc7 yeast is used as template. The sequence alignment is shown in FIG. 1.
ii) Sequences are retrieved from SwissProt using SRS6.0.4. Alignments are done using default parameters for Clustal X Software and are manually adjusted. The alignment of human Cdc34 with the other E2 enzymes is modified manually for the C-terminal helix. Consensus motifs of E1 enzymes are searched within the SRS Prosite database.
iii) Modelling of the zinc complex of BBIM—bis(2-benzimidazolyl)methane—is done in Sybyl 6.5.2 by simulated annealing with constraints for the metal coordination (Sybyl force field—no electrostatics). The ligand, the metal ion (dummy atom derived taking an Si atom as template), Tyr87, Cys93 and His98 of human Cdc34 are flexible; the rest of the protein is kept fixed.
iv) Data mining of e.g. a compound database is done using for example a 2D generic query such as the one given below; the hit list can then be refined to exclude undesired compounds (MW>500, etc.).

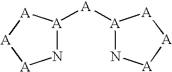

### 2. Preparation of Compounds

Temperatures are measured in degrees Celsius. Unless otherwise indicated, the reactions take place at room temperature (RT).

The short forms and abbreviations used have the following definitions:
h hour(s)
m.p. melting point
MS-ES mass spectroscopy (electron spray)
TFA trifluoroacetic acid
t_{R} retention times

If not otherwise indicated, the analytical HPLC conditions are as follows:

Thermo Finnigan SpectraSYSTEM, column: 250×4.6 mm packed with reversed-phase material (Uptisphere 5μ HDO C18, Interchrome). Detection by UV absorption at 216 nm. The retention times (t_{R}) are given in minutes. Flow rate: 2 ml/min. Solvents: A=0.1% aqueous TFA and B=0.1% TFA in acetonitrile. Gradient: linear solvent gradient from 2% B in A to 100% B over 10 min, then 3 min 100% B, flow rate 2.0 mL/min.

### Preparation of (3,5-dimethyl-pyrazol-1-yl)-(3-methyl-imidazo[2,1-b]thiazol-6-yl)-methanone

(Compound I)

A solution of 3-methyl-imidazo[2,1-b]thiazole-6-carboxylic acid (Abignente et al., Farmaco Ed. Sci. 1981, 36, 893-904, 91 mg, 0.5 mmol) is dissolved in thionyl chloride (3.6 ml) and heated at 80° C. for 1 h. The reaction mixture is diluted with diethyl ether (5 ml), the precipitate is filtered off and washed two times with diethyl ether. The crude residue is dissolved in dichloromethane and 3,5-dimethyl-1H-pyrazole (48 mg, 0.5 mmol) is added. The reaction mixture is refluxed for 3 h, diluted with dichloromethane, washed with sodium hydrogencarbonate 10%. The combined organic phases are dried over sodium sulphate and evaporated in vacuo. The residue is crystallized from dichloromethane/diethyl ether to afford the title compound as a white powder, m.p.=172-176° C., HPLC t_{R}: 6.45, (M+H)⁺=261.

### Preparation of Compounds II-IV

The following Compounds II-IV are prepared as described for the preparation of Compound I using the appropriate starting material:

### (3,5-Dimethyl-pyrazol-1-yl )-imidazo[2,1-b]thiazol-6-yl-methanone

(Compound II)

From imidazo[2,1-b]thiazole-6-carboxylic acid (Abignente et al., Farmaco Ed. Sci. 32, 735-746, 1977), m.p.=213-219° C., HPLC t_{R}: 5.87, (M+H)⁺=247.

### (6-Chloro-imidazo[1,2-a]pyridin-2-yl)-(3,5-dimethyl-pyrazol-1-yl)-methanone

(Compound III)

From 6-chloro-imidazo[1,2-a]pyridine-2-carboxylic acid, HPLC t_{R}: 6.20, (M+H)⁺=275.

### Benzo[d]imidazo[2,1-b]thiazol-2-yl-(3,5-dimethyl-pyrazol-1-yl)-methanone

(Compound IV)

From benzo[d]imidazo[2,1-b]thiazole-2-carboxylic acid, HPLC t_{R}: 8.10, (M+H)⁺=297.

### Preparation of (3,5-dimethyl-pyrazol-1-yl)-(3-propyl-imidazo[2,1-b]thiazol-6-yl)-methanone

(Compound V)

3,5-Dimethyl-1H-pyrazole (57 mg, 0.6 mmol) is added to a solution of 3-propyl-imidazo[2,1-b]thiazole-6-carboxylic acid (from procedure similar to Abignente et al., Farmaco Ed. Sci. 1981, 36, 893-904, 126 mg, 0.6 mmol), (3-dimethylamino-propyl)-ethyl-carbodiimide hydrochloride (172 mg, 0.9 mmol), and ethyl-diisopropyl-amine (0.31 ml, 1.2 mmol) in dichloromethane (10 ml). The reaction mixture is stirred 18 h at room temperature and evaporated in vacuo, the residue treated with aqueous sodium hydrogencarbonate, ethyl acetate and washed with aqueous sodium hydrogencarbonate. The combined organic phases are dried over sodium sulphate and evaporated in vacuo. The residue is crystallized from ethyl acetate to afford the title compound as a white powder, m.p.=170-173° C., HPLC t_{R}: 7.45, (M+H)⁺=289.

### 3. Materials and Methods for Thioester Assays

Abbreviations

CAMM Computer assisted molecular modelling
GST glutathione-S-transferase
NEM N-ethyl-maleimide
RT Room temperature
SAR Structure activity relationship
TEB Thioester buffer
TSQ N-(6-methoxy-8-quinolyl)-p-toluenesulfonamide

### 3.1. Labelling of ubiguitin with Europium

Bovine red blood cells ubiquitin (MW ˜8000; Sigma, U-6253) is labelled with Europium using the reagents supplied with the DELFIA® Europium labelling kit (Wallac, 1244-302) as described by the manufacturer. Typically, 2.5 mg ubiquitin (˜300 nmoles) are dissolved in 500 μl labelling buffer (50 mM NaHCO₃, 150 mM NaCl, pH=8.5) and added to 1 vial of DELFIA® Europium reagent (0.2 mg, 300 nmoles) in order to have an equimolar solution. After overnight incubation at RT, the labelled protein is separated from the remaining labelling reagent by gel filtration. To this purpose, a Sephadex G50-medium (Pharmacia) column (30×0.8 cm) equilibrated in elution buffer (50 mM Tris·HCl, pH=7.8, 150 mM NaCl) is used and 1 ml fractions are collected. Fractions containing the labelled material are identified by measuring the protein content by the Bradford method and by analysing Europium time resolved fluorescence using a Victor II® reader. Fractions containing Europium labelled ubiquitin (Eu—Ub) are pooled and stored as aliquots at −20° C. Analysis of Europium labelled ubiquitin and comparison with the standard supplied with the labelling kit shows that the pooled fractions (8 ml) contain ˜2 mg ubiquitin (˜250 nmoles) and 175 nmoles Europium, or 0.7 moles Europium/mole ubiquitin. This corresponds to 3.2×10⁹ counts/nmole ubiquitin.

### 3.2. Cloning, Expression and Purification of Human Ubiguitin-activating Enzyme E1 Containing N-terminally Fused GST (GST-E1)

Cloning of GST-E1: E1, isoform a, is cloned from a human uterus Quick Clone™ cDNA library (Clontech) by PCR (polymerase chain reaction). Primers are designed to cover the whole coding sequence (nucleotides 33-3209, see Genebank accession # X56976), and to allow subcloning in frame, downstream of GST and a Factor Xa cleavage site, into FastBacGSTx3 (Gibco BRL). The 5′-primer carries a BamHI cleavage site: 5′-T CGT GGG ATC CCC ATG TCC AGC TCG CCG CTG TCC AAG AAA-3′. The 3′-primer carries a HindIII cleavage site: 5′-TCG ACA AGC TTA GCG GAT GGT GTA TCG GAC ATA GGG AAC CTC-3′. The PCR mix contains 1 ng cDNA library, 200 μM dNTPs (dNTP: deoxynucleotide tri-phosphate), 200 nM of each primer and 2.5 U Takara ExTaq in 50 μl of 1× Takara PCR buffer (Takara Shuzo Co.). The PCR is initiated with 10 min denaturing at 94° C., followed by 30 amplification cycles (1 min denaturing at 94° C., 1 min annealing at 55° C., 3 min elongation at 72° C.) and is terminated with 10 min elongation at 72° C.

The PCR product of the expected size is subcloned as BamHI-HindIII insert into FastBacGSTx3, to yield FastBacGSTx3-E1. Its identity is confirmed by restriction mapping and sequencing. Comparison with the deposited sequence (Genebank accession # X56976) revealed the following amino acids changes: aa 148 ThrΠIIe; aa 190 GlyΠAsp; aa 434 GlnΠGlu; aa 1029 ArgΠHis.

Expression of GST-E1: Expression viruses for the GST-E1 expression construct are made according to the protocol supplied by Gibco BRL. In brief, FastBac transfer vectors containing GST-fused E1 (FastBacGSTx3-E1) are used to transform DH10Bac cells (Gibco BRL) by the standard heat shock method. Following 4 hours of incubation in S.O.C. medium, the bacteria are plated on agar plates containing the recommended concentrations of Bluo-Gal, IPTG (isopropyl thio-galactoside), Kanamycin, Tetracycline, and Gentamycin. After 24 hours at 37° C., colonies that have not had insertion of the fusion sequence into the viral genome (carried by the bacteria) turn blue. A single white colony is picked and used to inoculate a 3 ml overnight culture containing Kanamycin, Tetracycline, and Gentamycin. Viral DNA (bacmid) is isolated from the bacteria by standard plasmid mini prep procedures. High Five cells (Gibco BRL) are transfected in 25 cm² flasks with the viral DNA using the Cellfectin reagent and protocol supplied with the Bac-to-Bac kit (Gibco BRL). 48 hours after transfection, virus containing media is collected from the transfected cell culture and used (10× diluted) to infect cells at 70-80% confluency in 75 cm² flasks. After 48 hours the virus containing media is collected and the cells are used to test for protein expression by Western blotting. For large-scale protein expression, typically 10 P-150 plates are infected as above and cells are collected after 72 hours.

Purification of GST-E1: The cells harvested from ten P-150 plates are resuspended by vortexing in 50 ml lysis buffer (25 mM Tris·HCl, pH=7.5, 2 mM EDTA, 1% NP-40, 1 mM DTT, Complete™ (Roche) protease inhibitor cocktail) and incubated on ice for 30 min. After centrifugation at 2500×g for 20 min, the supernatant is incubated with 2 ml glutathione-Sepharose (Pharmacia) overnight at 4° C. on a rotor table. The latter is then packed in a column and washed extensively with 25 mM Tris·HCl, pH=7.5, 2 mM EDTA, 1 mM DTT. The GST-tagged protein is eluted using 50 mM Tris·HCl, pH=7.5, 1 mM reduced-glutathione, 1 mM DTT, 20% glycerol (87%). Fractions (1 ml) containing GST-fused-E1 are identified by SDS (sodium dodecyl sulfate) gel electrophoresis and Coomassie blue staining. The purified protein is stored at −70° C. in elution buffer.

### 3.3. Cloning, Expression and Purification of human Cdc34 Containing N-terminally Fused GST and a C-terminal FLAG-tag (GST-Cdc34-FLAG) or a C-terminal Strep-tag (GST-Ccd34-Strep)

Cloning of GST-Cdc34-FLAG and GST-Cdc34-Strep: Cdc34 is cloned from a human fetal brain Quick Clone™ cDNA library (Clontech) by PCR. Primers are designed to cover the whole coding sequence (nucleotides 187-894, see Genebank accession # L22005), and to allow subcloning in frame, downstream of GST and a Factor Xa cleavage site, into FastBacGSTx3. The 5′-primer carries a BamHI cleavage site: 5′-T CGT GGG ATC CCC ATG GCT CGG CCG CTA GTG CCC AGC TCG-3′. The 3′-primer carries an EcoRI cleavage site: 5′-CCT TTG AAT TCA GGA CTC CTC CGT GCC AGA GTC ATC CTC-3′. The PCR mix contains 1 ng cDNA library, 200 μM dNTPs, 200 nM of each primer and 2.5 U Takara ExTaq in 50 μl of 1× Takara PCR buffer (Takara Shuzo Co.). The PCR is initiated with 10 min denaturing at 94° C., followed by 30 amplification cycles (1 min denaturing at 94° C., 1 min annealing at 55° C., 1 min elongation at 72° C.) and is terminated with 10 min elongation at 72° C.

The PCR product of the expected size is subcloned as BamHI-EcoRI insert into FastBacGSTx3 to yield FastBacGSTx3-Cdc34. Its identity can be confirmed by restriction mapping and sequencing.

Using the clone obtained above as a template, a C-terminal FLAG-tag (DYKDDDDK) version is constructed by PCR using the same 5′-primer, suitable for subcloning into FastBacGSTx3, and the following 3′-primer containing an EcoRI site: 5′-CC TTG AAT TCA CTT GTC GTC GTC GTC CTT GTA GTC GGA CTC CTC CGT GCC AGA-3′. The PCR is performed as above using 2.5 U Pfu (Stratagene). The product is cloned into FastBacGSTx3 to yield FastBacGSTx3-Cdc34-FLAG. Its identity can be confirmed by sequencing.

Similarly, a C-terminal Strep-tag (AWRHPQFGG) version is constructed using the same 5′-primer and the following 3′-primer containing an EcoRI site: 5′-CC TTG AAT TCA ACC ACC GAA CTG CGG GTG ACG CCA AGC GGA CTC CTC CGT GCC AGA-3′. The PCR is performed as above using 2.5 U Pfu (Stratagene). The product is cloned into FastBacGSTx3 to yield FastBacGSTx3-Cdc34-Strep. Its identity is confirmed by sequencing.

Expression of GST-Cdc34-FLAG and GST-Cdc34-Strep: Expression viruses for the GST-Cdc34-FLAG and GST-Cdc34-Strep expression constructs are made according to the protocol supplied by Gibco BRL (see also above under ‘Expression of GST-E1’).

Purification and Factor Xa cleavage of GST-Cdc34-FLAG and GST-Cdc34-Strep: The cells harvested from ten P-150 plates are resuspended by vortexing in 50 ml lysis buffer (25 mM Tris·HCl, pH=7.5, 2 mM EDTA, 1 mM DTT, 1 mM PMSF) and incubated on ice for 30 min. After centrifugation at 8000×g for 20 min, the supernatant is incubated with 2 ml glutathione-Sepharose (Pharmacia) overnight at 4° C. on a rotor table. The latter is then packed in a column and washed extensively with 25 mM Tris·HCl, pH=7.5, 100 mM NaCl, 0.5 mM DTT. Glutathione-Sepharose is washed with Factor Xa buffer (50 mM Tris·HCl, pH=8.0, 100 mM NaCl, 1 mM CaCl₂) and resuspended in 2 ml of the same buffer. Cdc34-FLAG and Cdc34-Strep are subsequently cleaved off the GST-fusion by adding 100 μg of Factor Xa (Roche) to the glutathione-Sepharose suspension and incubating for 2 hours at RT on a rotor table. After centrifugation, the supernatants containing Cdc34-FLAG or Cdc34-Strep are collected and analysed by SDS gel electrophoresis and Coomassie blue staining. The purified proteins are stored at −70° C. upon addition of 0.5 mM DTT and 10% glycerol (87%).

### 3.4. Cloning, Expression and Purification of Human Ubiguitin-conjugating Enzyme UbcH5 Containing N-terminally Fused GST and a C-terminal Strep-tag (GST-UbcH5-Strep)

Cloning of GST-UbcH5-Strep: UbcH5 is cloned from a human placenta Quick Clone™ cDNA library (Clontech) by PCR. Primers bam2_Ub5: 5′-TCAGGGATCCCCATGGCGCTGAAGAGGATTC-3′ and Ub5_Eco: 5′-CTCGGAATTCTTACATTGCATATTTC-3′ are designed to cover the whole coding sequence (nucleotides 4-459, see Genebank accession # X78140), and in order to clone UbcH5 as a GST fusion protein in the vector pFastBacGSTx3 between BamHI and EcoRI sites. Using the clone obtained above as a template, a C-terminal strep-tag (AWRHPQFGG) version is constructed by PCR using the same 5′-primer, and the following 3′-primer containing an EcoRI site: UbcH5-Strep: 5′-CCTTGAATTCAACCACCGAACTGCGGGTGACGCCAAGCCATTGCATATTTCTGAGTCC-3′. The resulting fragment is restriction digested (BamHI-EcoRI) and reinserted into pFastBacGSTx3. For all reactions PCR mix contains 1 ng cDNA library or 20 ng template-plasmid, 200 pM dNTPs, 200 nM of each primer and 2.5 U Takara ExTaq in 50 μl of 1× Takara PCR buffer (Takara Shuzo Co.). The PCR is initiated with 10 min denaturing at 94° C., followed by 30 amplification cycles (1 min denaturing at 94° C., 1 min annealing at 55° C., 1 min elongation at 72° C.) and is terminated with 10 min elongation at 72° C.

For all constructs, identity of UbcH5 and checking of in-frame fusions can be confirmed by restriction mapping, sequencing and comparison with the deposited sequence for UbcH5 (Genebank accession # X78140) and vectors' sequences provided by the vendor.

Expression of GST-UbcH5-Strep: Expression viruses for the GST-UbcH5-Strep expression construct are made according to the protocol supplied by Gibco BRL (see also above under ‘Expression of GST-E1’).

Purification and Factor Xa cleavage of GST-UbcH5-Strep: Purification of UbcH5-Strep is done as described above under ‘Purification and Factor Xa cleavage of GST-Cdc34-FLAG’.

### 3.5. Cloning of Human Ubiguitin-conjugating Enzyme UbcH10 Containing N-terminally Fused GST and a C-terminal Strep-tag (GST-UbcH10-Strep)

Cloning of GST-UbcH10-Strep: UbcH10 is cloned from a human fetal brain Quick Clone™ cDNA library (Clontech) by PCR. Primers are designed to cover the whole coding sequence (nucleotides 41-580, see Genebank accession # U73379), and to allow subcloning in frame, downstream of GST and a Factor Xa cleavage site, into FastBacGSTx3. The 5′-primer carries a BamHI cleavage site: 5′-TCG TGG GAT CCC CAT GGC TTC CCA AAA CCG CGA CCC AGC C-3′. The 3′-primer carries an EcoRI cleavage site: 5′-CCT TTG MT TCA GGG CTC CTG GCT GGT GAC CTG CTT TGA-3′. The PCR mix contains 1 ng cDNA library, 200 μM dNTPs, 200 nM of each primer and 2.5 U Takara ExTaq in 50 μi of 1× Takara PCR buffer (Takara Shuzo Co.). The PCR is initiated with 10 min denaturing at 94° C., followed by 30 amplification cycles (1 min denaturing at 94° C., 1 min annealing at 55° C., 1 min elongation at 72° C.) and is terminated with 10 min elongation at 72° C.

The PCR product of the expected size is subcloned as BamHI-EcoRI insert into FastBacGSTx3 to yield FastBacGSTx3-UbcH10. Its identity can be confirmed by restriction mapping, sequencing and comparison with the deposited sequence (Genebank accession # U73379).

Using the clone obtained above as a template, a C-terminal strep-tag (AWRHPQFGG) version is constructed by PCR using the same 5′-primer, suitable for subcloning into FastBacGSTx3, and the following 3′-primer containing an EcoRI site: 5′-CCT TGA ATC ACC ACC GAA CTG CGG GTG ACG CCA AGC GGG CTC CTG GCT GGT GAC C-3′. The PCR is performed as above using 2.5 U Pfu (Stratagene). The product is cloned into FastBacGSTx3 to yield FastBacGSTx3-UbcH10-Strep. Its identity can be confirmed by sequencing.

Expression of GST-UbcH10-Strep: Expression viruses for the GST-UbcH10-Strep expression construct are made according to the protocol supplied by Gibco BRL (see also above under ‘Expression of GST-E1’).

Purification and Factor Xa cleavage of GST-UbcH10-Strep: Purification of UbcH10-Strep is done as described above under ‘Purification and Factor Xa cleavage of GST-Cdc34-FLAG’.

### 3.6. Thioester Assays

The assays measuring the formation of the thioester intermediate between ubiquitin and the ubiquitin conjugating enzymes Cdc34 (SwissProt P49427) UbcH5 (SwissProt P51668) and UbcH10 (SwissProt O00762) are described below.

Cdc34 thioester assay: Here, we describe an assay, based on time resolved fluorescence, to monitor the formation of the thioester intermediate between ubiquitin and the ubiquitin-conjugating enzyme Cdc34. The assay is aimed at screening for inhibitors of Cdc34 thioester formation and requires Eu-labelled ubiquitin (Eu—Ub), GST-E1 and Cdc34-FLAG.
Mix: In non-absorbing plates
   10 μl Test compound 5×conc. in 5% DMSO
   30 μl Cdc34-FLAG (20.8 nM) in 1×TEB (no ATP)
      Incubate 10 min at RT
Add: 10 μl Eu—Ub (31.2 nM), GST-E1 (125 nM) in 1×TEB+ATP (Pre-incubated 10 min at RT).
   Incubate 30 min at RT
Final Concentrations: Test compound 50/12.5/3.13/0.78/0.2/0.05 μM
   Cdc34-FLAG 12.5 nM
   Eu—Ub 6.25 nM
   GST-E1 25 nM
Transfer: 45 μl To streptavidin coated plate containing 50 μl / well Superblock−TBS+1 mM NEM+100 nM biotinylated anti-FLAG antibody
   Incubate 2 hours at RT
   Wash 6×200 μl/well with 1× Wallac Wash Solution
   Add 200 μl/well Wallac Enhancer Solution
   Mix 5 min
   Read
Positive Control: no test compound, 10 μl 5% DMSO
Negative Control: no Cdc34-FLAG, 30 μl 1×TEB (no ATP)

UbcH5 and UbcH10 thioester assays: Here, we describe an assay, based on time resolved fluorescence, to monitor the formation of the thioester intermediate between ubiquitin and the ubiquitin-conjugating enzymes UbcH5, respectively UbcH10. The assay is aimed at screening for inhibitors of UbcH5 or UbcH10 thioester formation and requires Eu-labelled ubiquitin, GST-E1 and UbcH5-Strep, respectively UbcH10-Strep.
Mix: In non-absorbing plates
   10 μl Test compound 5×conc. in 5% DMSO
   30 μl UbcH5-Strep/UbcH10-Strep (20.8 nM) in 1×TEB (no ATP)
      Incubate 10 min at RT
Add: 10 μl Eu—Ub (31.2 nM), GST-E1 (125 nM) in 1×TEB+ATP (Pre-incubated 10 min at RT).
   Incubate 30 min at RT
Final Concentrations: Test compound 50/12.5/3.13/0.78/0.2/0.05 μM
   UbcH5-Strep/UbcH10-Strep 12.5 nM
   Eu—Ub 6.25 nM
   GST-E1 25 nM
Transfer: 45 μl To streptavidin coated plate containing 50 μl/ well Superblock−TBS+1 mM NEM
   Incubate 2 hours at RT
   Wash 6×200 μl/well with 1× Wallac Wash Solution
   Add 200 μl/well Wallac Enhancer Solution
   Mix 5 min
   Read
Positive Control: no test compound, 10 μl 5% DMSO
Negative Control: no UbcH5-Strep/UbcH10-Strep, 30 μl 1×TEB (no ATP)
   Buffers:
10×TEB 1.0 M Tris·HCl, pH=7.5
   1.0 M NaCl
   100 mM MgCl₂
   2.5 mM DTT
10×TEB+ATP as above+40 mM′ATP (Sigma # A-238)
Adjust volumes of assay mix with 10 mM Tris·HCl, pH=7.5
   Materials:
Non absorbing plates (Microtec # 651 170)
SuperBlock-TBS (Pierce # 37535)
Streptavidin coated plate (Pierce # 15124)
NEM: N-Methyl-maleimide (Fluka # 04259)
Anti-FLAG antibody (Sigma # F-9291)
Wallac Wash Solution (Wallac # 1244-114)
Wallac Enhancement Solution (Wallac # 1244-105)
Victor II Reader (Wallac)

Cdc34 thioester assay in the presence of the chelating agents phenantroline or TSQ In order to deplete Cdc34 from zinc prior to measuring the inhibitory activity of the test compounds, the Cdc34 thioester assay described above was modified as follows.
Mix: In non-absorbing plates on ice
   10 μl Cdc34-FLAG (62.5 nM) in 1×TEB (no ATP)
   10 μl 5× Phenanthroline/5×TSQ/buffer
   10 μl 5×ZnCl₂/buffer
      Incubate 10 min on ice
Add: 10 μl Test compound 5×conc. in 5% DMSO
   10 μl Eu—Ub (31.2 nM), GST-E1 (125 nM) in 1×TEB+ATP (Pre-incubated 10 min at RT).
      Incubate 30 min at RT
Phenanthroline (Sigma # P-9375)
TSQ (Molecular Probes # M-688)

### 3.7. IC₅₀ Determination

The IC₅₀ is defined as 50% inhibition of E2-ubiquitin thioester formation, estimated as the difference in fluorescence obtained in the presence or absence of E2 (Cdc34, UbcH5 or UbcH10).

### 4. Results

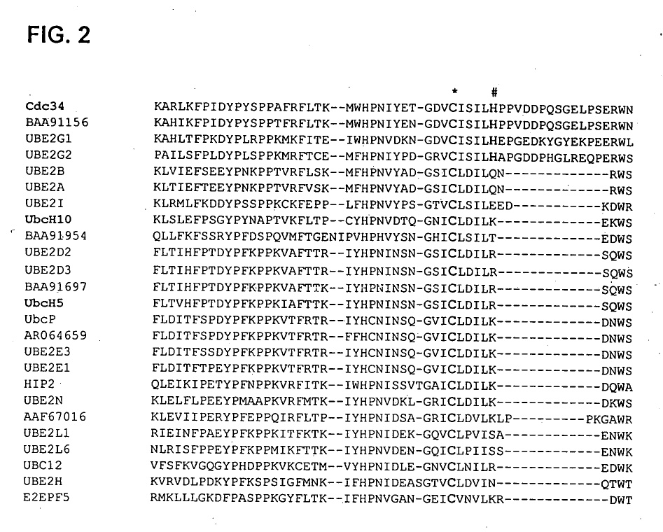

Comparison of the active site of 25 canonical E2 sequences available from the public domain (e.g. SwissProt database) revealed that the cysteine-histidine pair is peculiar to Cdc34 and its three most closely related ubiquitin conjugating enzymes (FIG. 2).

In order to test whether zinc is specifically associated with Cdc34, the zinc present in the Cdc34 protein solution, in the related E2s, UbcH5 and UbcH10, protein solutions, as well as in their storage buffer was measured by atomic absorption spectroscopy analysis at Solvias AG (Basel, Switzerland). All three E2 proteins were expressed using the baculo virus system and purified according to the same procedure (see above). At difference with Cdc34, UbcH5 and UbcH10 do not have a cysteine-histidine pair at the active site; the histidine residue is substituted by an arginine, respectively a lysine residue (FIG. 2). Strikingly, only the Cdc34 protein preparations showed the presence of zinc at significant levels (Table 1), while no zinc above detection level (at least 100-200 fold less) was found associated with UbcH5 and UbcH10. Other metals, such as cobalt, cadmium, copper, iron and mangan were not detected in the Cdc34-Strep preparation (data not shown).

TABLE 1

| | Moles Zinc/Moles Protein | | |
|---|---|---|---|
| | Exp. 1 | Exp. 2 | Exp. 3 |
| Cdc34-Strep | 0.72 | 0.71 | 0.78 |
| Cdc34-FLAG | | | 0.60 |
| UbcH5-Strep | | <0.07 | |
| UbcH10-Strep | | <0.03 | <0.03 |

Table 1: Atomic absorption spectroscopy is used to measure the content of zinc in the indicated purified protein solutions. Protein concentrations ranged between 20 and 50 μM, allowing clear detection of zinc by this technique, if present in a significant molar ratio. This was found to be the case for Cdc34 in the Strep- or FLAG-tagged version, but not for the related ubiquitin conjugating enzymes UbcH5 and UbcH10.

Compound V was selected by CAMM as a potential Cdc34 zinc-mediated inhibitor, assuming that it would specifically bind at the active site of Cdc34, by coordinating a zinc atom together with the cysteine-histidine pair. Compound V and a derivative thereof (Compound I) were tested for inhibition of Cdc34, UbcH5 and UbcH10 in their respective thioester assays (see above for description of the thioester assays). Both compounds were found to be potent and specific inhibitors of Cdc34 (Table 2). Thus, their inhibitory activity and absolute selectivity correlate with the fact that zinc is found associated with Cdc34, but not with UbcH5 or UbcH10. The same it true for Compounds II, III and IV, which are derivatives of Compound V (for the preparation of Compounds I-V see above). Compounds II, III and IV exhibit IC₅₀ values≦1 μM as determined in the Cdc34 thioester assays described herein.

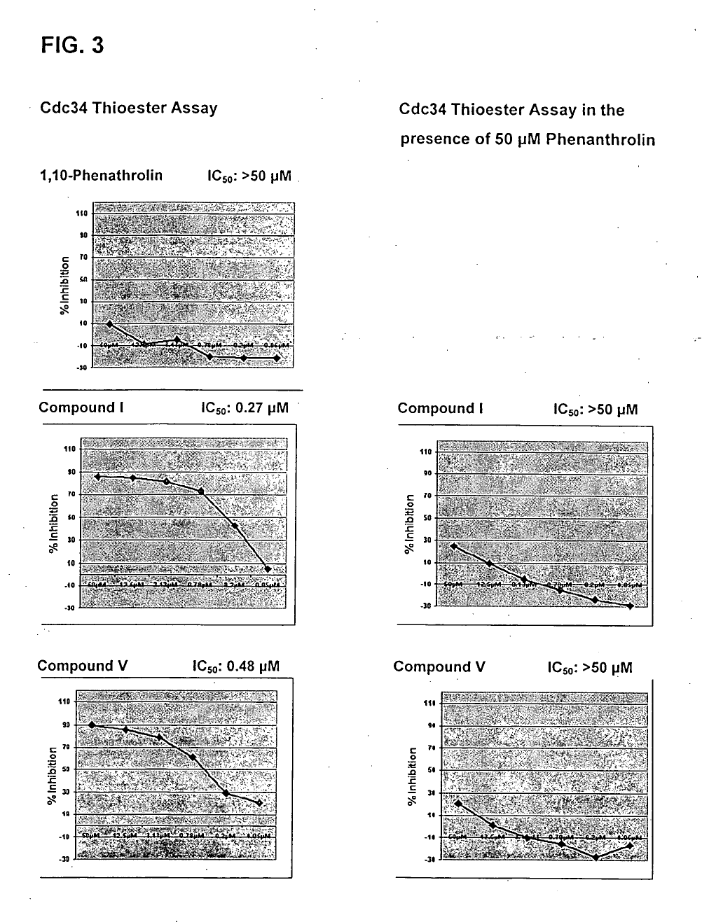

In order to gain further evidence in support of the idea that the inhibitory activity of Compound I and V depends on the zinc atom associated with Cdc34, we tested whether depletion of the metal ion by a broad specificity chelating agent (phenanthroline) would abolish their ability to inhibit Cdc34 thioester formation. As shown in FIG. 3 (left panel), phenathroline does not affect per se the ability of Cdc34 to undergo thioester formation with ubiquitin, however both Compound I and Compound V inhibit Cdc34 thioester formation with IC₅₀ values in the range of those reported in Table 2. On the other hand, when Cdc34 was pretreated with 50 μM phenanthroline, the inhibitory activity of both Compound I and Compound V was completely abolished (FIG. 3; right panel).

TABLE 2
IC₅₀ values for Compound I and Compound V as determined in the Cdc34, UbcH5 and UbcH10
thioester assays

| | Cdc34-FLAG | | | UbcH5-Strep | | UbcH10-Strep | |
|---|---|---|---|---|---|---|---|
| | IC₅₀ [μM] | n | stdv | IC₅₀ [μM] | n | IC₅₀ [μM] | n |
| 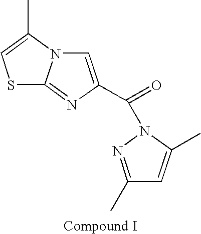 | 0.39 | 5 | 0.17 | >50 | 2 | >50 | 2 |
| 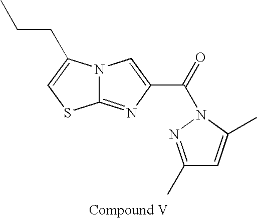 | 0.95 | 5 | 0.29 | >50 | 2 | >50 | 2 |

Table 2: n: number of independent experiments; stdv: standard deviation.

Interestingly, when Compound I and V were added to Cdc34 before phenantroline, the latter failed to abolish the inhibitory activity of the compounds (data not shown), suggesting that Compound I and V compete with phenanthrolin for binding to the same metal (i.e. zinc). Since phenanthroline is not specific for zinc, we then tested whether a more zinc-specific chelator TSQ (Frederickson et al., 1987) could also abolish the activity of Compound I. Further, we also tested whether, upon pretreatment of Cdc34 with phenanthroline or TSQ, the addition of zinc to the experimental system would restore the inhibitory activity of Compound I. Indeed, both phenathroline and TSQ were found to reduce the inhibitory potential of Compound I in a dose-dependent manner and the re-addition of zinc was shown to restore inhibition of Cdc34 thioester formation by the compound (Table 3).

Taking together, these observations demonstrate that Cdc34 binds zinc and that compounds can be designed to potently and specifically inhibit this enzyme in a zinc-mediated manner.

TABLE 3
Table 3: IC₅₀ values for Compound I as determined
in the Cdc34 thioester assay upon pre-treatment of the enzyme
with increasing concentrations of phenanthroline or TSQ
and upon re-addition of increasing concentrations of zinc.
Compound I IC₅₀ - μM

| ZnCl₂ | 0 μM | 2 μM | 10 μM |
|---|---|---|---|
| | 1,10-Phenanthroline | | |
| 0 nM | 0.74 | 1.4 | 8.8 |
| 100 nM | 0.55 | 0.64 | 4.6 |
| 200 nM | 0.36 | 0.71 | 1.38 |
| | TSQ | | |
| 0 nM | 0.64 | 4.2 | 10.6 |
| 100 nM | 0.6 | 0.95 | 3.3 |
| 200 nM | 0.7 | 1.3 | 0.6 |

1: A method of searching for compounds which inhibit the activity of the ubiquitin conjugating enzyme Cdc34 in a zinc-mediated manner, which method comprises identifying compounds capable of coordinating zinc together with the active site cysteine-histidine residues of Cdc34 using a computer assisted molecular modelling-based approach.

2: A method of screening for compounds which inhibit the activity of the ubiquitin conjugating enzyme Cdc34 in a zinc-mediated manner, which method comprises determining the capacity of a compound to inhibit the activity of Cdc34 in the presence of zinc [Cdc34(+)], determining the capacity of the compound to inhibit the activity of Cdc34 that is depleted of zinc [Cdc34(−)], comparing both capacities and selecting compounds which inhibit the activity of Cdc34(+) to a greater extent than Cdc34(−).

3: A compound obtainable by the method of claim 2, or a pharmaceutically acceptable salt of such a compound.

4: A compound according to claim 3, selected from the group consisting of (3,5-dimethyl-pyrazol-1-yl)-(3-methyl-imidazo[2,1-b]thiazol-6-yl)-methanone, (3,5-dimethyl-pyrazol-1-yl)-imidazo[2,1-b]thiazol-6-yl-methanone, (6-chloro-imidazo[1,2-a]pyridin-2-yl)-(3,5-dimethyl-pyrazol-1-yl)-methanone, benzo[d]imidazo[2,1-b]thiazol-2-yl-(3,5-dimethyl-pyrazol-1-yl)-methanone and (3,5-dimethyl-pyrazol-1-yl)-(3-propyl-imidazo[2,1-b]thiazol-6-yl)-methanone, or a pharmaceutically acceptable salt of such a compound.

5: A method of treating a mammalian subject suffering from a disease which responds to an inhibition of the activity of the ubiquitin conjugating enzyme Cdc34, which method comprises administering to the subject suffering from said disease a compound of claim 3, or a pharmaceutically acceptable salt of such a compound, in an amount effective against said disease.

6: The method of claim 5, wherein the disease to be treated is a proliferative disease.

7: The method of claim 6, wherein the proliferative disease is a tumour disease.

8: A compound of claim 3, or a pharmaceutically acceptable salt of such a compound, for use in a method for the therapeutic treatment of the human or animal body.

9: A pharmaceutical composition comprising a compound of claim 3, or a pharmaceutically acceptable salt of such a compound, together with a pharmaceutically acceptable carrier.

10: A pharmaceutical composition for the treatment of a disease which responds to an inhibition of the activity of the ubiquitin conjugating enzyme Cdc34 in a mammalian subject, comprising, in a dose effective against said disease, a compound of claim 3, or a pharmaceutically acceptable salt of such a compound, together with a pharmaceutically acceptable carrier.

11: Use of a compound of claim 3, or a pharmaceutically acceptable salt of such a compound, for the preparation of a pharmaceutical composition for use in the treatment of a disease which responds to an inhibition of the activity of the ubiquitin conjugating enzyme Cdc34.

12: Use of a compound of claim 3, or a pharmaceutically acceptable salt of such a compound, in the treatment of a disease which responds to an inhibition of the activity of the ubiquitin conjugating enzyme Cdc34.


SEQUENCE LISTING (14 sequences)

SEQ ID NO: 1 - DNA, 40 bp, artificial sequence
  misc_feature (1)..(40): GST-E1-5′-primer carries a BamHI cleavage site
tcgtgggatccccatgtccagctcgccgctgtccaagaaa

SEQ ID NO: 2 - DNA, 42 bp, artificial sequence
  misc_feature (1)..(42): GST-E1 - 3′-primer carries an HindIII cleavage site
tcgacaagcttagcggatggtgtatcggacatagggaacctc

SEQ ID NO: 3 - DNA, 40 bp, artificial sequence
  misc_feature (1)..(40): GST-Cdc34-5′-primer carrying a BamHI cleavage site
tcgtgggatccccatggctcggccgctagtgcccagctcg

SEQ ID NO: 4 - DNA, 39 bp, artificial sequence
  misc_feature (1)..(39): GST-Cdc34-3′-primer carrying an EcoRI cleavage site
cctttgaattcaggactcctccgtgccagagtcatcctc

SEQ ID NO: 5 - DNA, 53 bp, artificial sequence
  misc_feature (1)..(53): GST-Cdc34-FLAG -3′-primer containing an EcoRI site
ccttgaattcacttgtcgtcgtcgtccttgtagtcggactcctccgtgccaga

SEQ ID NO: 6 - DNA, 56 bp, artificial sequence
  misc_feature (1)..(56): GST-Cdc34-Strep-3′-primer containing an EcoRI site
ccttgaattcaaccaccgaactgcgggtgacgccaagcggactcctccgtgccaga

SEQ ID NO: 7 - DNA, 31 bp, artificial sequence
  misc_feature (1)..(31): Primers bam2_Ub5
tcagggatccccatggcgctgaagaggattc

SEQ ID NO: 8 - DNA, 26 bp, artificial sequence
  misc_feature (1)..(26): primer Ub5_Eco
ctcggaattcttacattgcatatttc

SEQ ID NO: 9 - DNA, 58 bp, artificial sequence
  misc_feature (1)..(58): UbcH5-Strep
ccttgaattcaaccaccgaactgcgggtgacgccaagccattgcatatttctgagtcc

SEQ ID NO: 10 - DNA, 40 bp, artificial sequence
  misc_feature (1)..(40): GST-UbcH10-Strep - 5′-primer carrying a BamHI cleavage site
tcgtgggatccccatggcttcccaaaaccgcgacccagcc

SEQ ID NO: 11 - DNA, 39 bp, artificial sequence
  misc_feature (1)..(39): GST-UbcH10-Strep-3′-primer carries an EcoRI cleavage site
cctttgaattcagggctcctggctggtgacctgctttga

SEQ ID NO: 12 - protein, 9 aa, artificial sequence
  misc_feature (1)..(9): C-terminal strep-tag
AWRHPQFGG

SEQ ID NO: 13 - DNA, 55 bp, artificial sequence
  misc_feature (1)..(55): 3′-primer containing an EcoRI site-GST-UbcH10- Tag
ccttgaatcaccaccgaactgcgggtgacgccaagcgggctcctggctggtgacc

SEQ ID NO: 14 - DNA, 8 bp, artificial sequence
  misc_feature (1)..(8): C-terminal FLAG-tag
dykddddk